(19) United States
(12) Patent Application Publication
(10) Pub. No.: US 2006/0122465 A1
(43) Pub. Date: Jun. 8, 2006

(54) Methods and systems for generating diagnostic algorithms based on questionnaires

(76) Inventors: Philippe Bastien, Charenton Le Pont (FR); Roland Jourdain, Meudon La Foret (FR); Olivier De LaCharriere, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/517,391

(22) PCT Filed: Jun. 12, 2003
(86) PCT No.: PCT/IB03/02788

§ 371 Date: Aug. 23, 2005

Related U.S. Application Data
(60) Provisional application No. 60/387,918, filed on Jun. 13, 2002.

(51) Int. Cl. A61B 5/00

(52) U.S. Cl. 600/300; 128/920

(57) ABSTRACT

Methods and systems of the present invention generally relate to questionnaires and diagnostic algorithms. Features and principles of the present invention may include a method of generating a diagnostic algorithm. The method may involve receiving information reflecting a plurality of individuals' answers to questions, performing an analysis on the received information to generate a synthetic variable, and generating a diagnostic algorithm from at least the synthetic variable using a tree segmentation technique.

### CLAIM FOR PRIORITY

This application claims benefit of priority to U.S. Provisional Patent Application No. 60/387,918 filed on Jun. 13, 2002, which is incorporated herein by reference.

### BACKGROUND

1. Technical Field

The present invention generally relates to questionnaires and diagnostic algorithms.

2. Related Art

Traditional diagnostic methods are often inconvenient, labor intensive, and expensive because they require in-depth analysis by trained experts. In the realm of personal diagnosis, for example, a subject suffering from an illness or other health condition typically has to visit a medical professional to diagnose his/her condition. This may require scheduling an appointment with the professional, traveling to the professional's office, waiting to be seen by the professional, finally being examined, and possibly returning to the office for subsequent examinations. The examination itself may require the subject to answer many questions and undergo a large battery of tests before a proper diagnosis is obtained. Many times this process may require more time and effort, and be more expensive than the subject is willing to accept. Accordingly, the subject may choose to not bother with the diagnosis. Thus, a convenient mechanism for the subject to obtain a diagnosis from a remote location without answering an overwhelming number questions would be beneficial.

Further, it would be equally beneficial in the realm of dermatological, cosmetic, and beauty diagnoses for a subject to obtain a convenient and less time-consuming diagnosis, especially from a remote location. For example, in the case of a customer at a cosmetic counter, it would be convenient to allow the customer to be able to determine which cosmetic would best suit her without her having to answer an unduly large number questions. This would ensure an efficient and pleasant cosmetic selection process for the customer while providing her with the most appropriate cosmetic product for her needs.

It will become apparent in the following description that many aspects of the present invention have applicability in fields other than those described above. Accordingly, the background discussion should be considered to be exemplary of a few of many possible background issues that could be addressed.

### SUMMARY OF EXEMPLARY EMBODIMENTS

Methods, systems, and articles of manufacture of the present invention may generate a diagnostic algorithm that provides a diagnosis using a reduced set of discriminating questions.

One exemplary aspect of the invention relates to a diagnostic method comprising providing a first question, receiving first information reflecting an answer to the first question, selecting a second question according to the first information and according to a diagnostic algorithm generated using at least one of a multivariate analysis and a tree segmentation technique, providing the second question, receiving second information reflecting an answer to the second question, and determining a diagnosis according to the diagnostic algorithm.

A second exemplary aspect of the invention relates to a diagnostic method, comprising receiving a first question, sending first information reflecting an answer to the first question, receiving a second question, wherein the second question is a question selected according to the first information and according to a diagnostic algorithm generated using at least one of a multivariate analysis and a tree segmentation technique, and sending second information reflecting an answer to the second question, wherein a diagnosis is determined according to the diagnostic algorithm.

A third exemplary aspect of the invention relates to a method of generating a diagnostic algorithm, comprising receiving information reflecting a plurality of individuals' answers to questions, performing an analysis on the received information to generate a synthetic variable, and generating a diagnostic algorithm from at least the synthetic variable using a tree segmentation technique.

Other exemplary aspects of the invention include computer systems and/or computer program products used to implement the above exemplary methods.

Additional embodiments and aspects of the invention are set forth in the detailed description which follows, and in part are obvious from the description, or may be learned by practice of methods, systems, and articles of manufacture consistent with the present invention. It is understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several aspects of the invention and together with the description, serve to explain the principles of the invention. In the drawings:

FIG. 1 illustrates an exemplary method of generating a diagnostic algorithm consistent with features and principles of the present invention;

FIG. 2 illustrates an exemplary questionnaire consistent with features and principles of the present invention;

FIG. 3 illustrates an exemplary method of presenting a questionnaire consistent with features and principles of the present invention;

FIG. 4 illustrates an exemplary method of performing an analysis to generate a synthetic variable consistent with features and principles of the present invention;

FIG. 5 illustrates an exemplary diagnostic algorithm consistent with features and principles of the present invention;

FIGS. 6A and 6B illustrate an exemplary diagnostic method consistent with features and principles of the present invention;

FIG. 7 illustrates an exemplary display on a electronic device consistent with features and principles of the present invention;

FIG. 8 illustrates another exemplary diagnostic method consistent with features and principles of the present invention;

FIG. 9 displays exemplary incidences of positive responses to questions in the questionnaire consistent with features and principles of the present invention;

FIG. 10 illustrates a second exemplary diagnostic algorithm consistent with features and principles of the present invention;

FIG. 11 displays exemplary comparisons between subpopulations consistent with features and principles of the present invention;

FIG. 12 illustrates an exemplary typology constructed by factorial analysis consistent with features and principles of the present invention;

FIG. 13 illustrates exemplary average profiles of positive responses consistent with features and principles of the present invention;

FIG. 14 illustrates exemplary correlations between questions and sub-topics consistent with features and principles of the present invention;

FIG. 15 illustrates exemplary correlations between various sub-topics consistent with features and principles of the present invention;

FIG. 16 illustrates an exemplary variances of components in a principal component analysis consistent with features and principles of the present invention;

FIG. 17 illustrates exemplary projections of sub-topics onto a factorial plane consistent with features and principles of the present invention;

FIG. 18 illustrates an exemplary projection of individuals onto a first factorial plane consistent with features and principles of the present invention;

FIG. 19 illustrates an exemplary distribution of individuals into sensitivity classes consistent with features and principles of the present invention;

FIG. 20 illustrates a third exemplary diagnostic algorithm consistent with features and principles of the present invention;

FIGS. 21 and 22 respectively illustrate a histogram and frequency of overall sensitivity scores consistent with features and principles of the present invention;

FIG. 23 illustrates a fourth exemplary diagnostic algorithm consistent with features and principles of the present invention; and

FIG. 24 illustrates an exemplary diagnostic system consistent with features and principles of the present invention.

### DETAILED DESCRIPTION

Reference is now made in detail to exemplary aspects of the invention, examples of which are illustrated in the accompanying drawings. Wherever possible, the same reference numbers are used throughout the drawings to refer to the same or like parts.

Methods and systems of the present invention generally relate to questionnaires and diagnostic algorithms. Starting with a list of questions relevant to a diagnosis, facilitators may present the questions to a sample number of individuals. The facilitators may collect the individuals' answers to the questions and may perform a multivariate analysis on the answers to the questionnaire with a goal of giving a synthetic score relevant to each individual. This synthetic score (i.e., synthetic variable) may summarize for each individual all the information of an investigated topic. From this analysis, the facilitators may generate a reduced set of questions for presenting to a subject and a corresponding algorithm to diagnose the subject. The reduced set of questions and algorithm may provide a fast and accurate method of diagnosing the subject without requiring the subject to answer all the questions on the original list. The diagnosis may include one or more of a dermatological diagnosis (e.g., acne), a keratinous diagnosis (e.g., an evaluation of a subject's hair and/or nails), a beauty diagnosis, a cosmetic diagnosis (e.g., an overall sensitivity of a subject's skin), or any other diagnosis of interest.

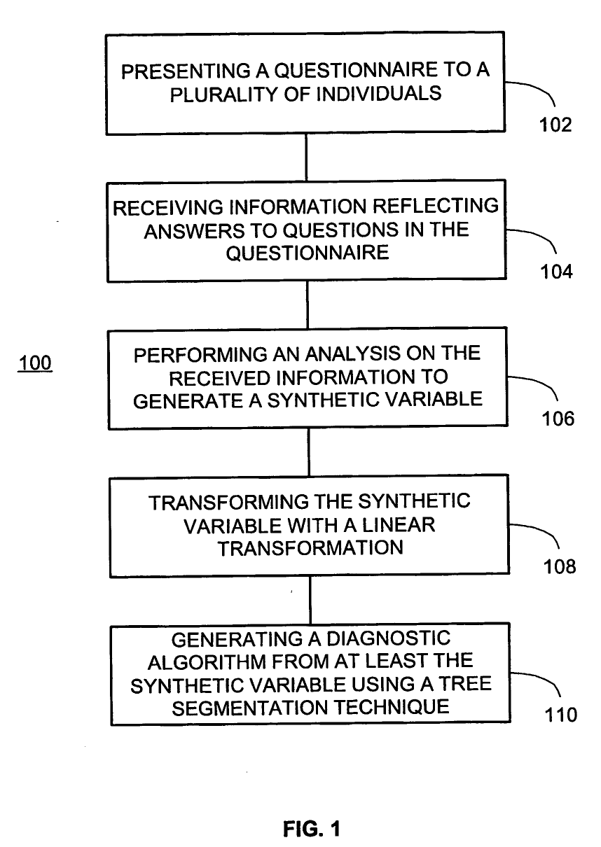

Features and principles consistent with the present invention may include a method 100 of generating a diagnostic algorithm, as illustrated in FIG. 1. Method 100 may comprise presenting a questionnaire to a plurality of individuals (step 102). “Presenting” may include sending questions in the questionnaire over a network (e.g., an internet, intranet, wireless network, wired network, postal network, courier network, etc.), at a kiosk, in a waiting room, at the individuals' homes, at a point-of-sale (e.g., a product-selling store, a service-providing location, website, etc.), or in any other manner. “Presenting” may include displaying, playing, or transmitting the questions to the individuals via a computer, a telephone, a mobile phone, a television set, a personal organizer, or any other suitable electronic communication device. “Presenting” may include making the questions available for access or providing the questions indirectly to the individuals via one or more third parties (e.g., a third-party polling service, a mail/paper distributor, etc.).

The questions may include inquiries about various sub-topics relating to atopy, self-perceived sensitivity, cosmetic reactivity, environmental reactivity, vascular reactivity, skin characteristics, skin (including scalp) conditions, keratin characteristics, keratin conditions, or any other matter of interest. Skin conditions may include at least one of greasy skin, dry skin, aging skin, wrinkled skin, dirty skin, marked skin, flask skin, squeamish skin, sensitive skin, skin phototype, cellulitic skin, skin discoloring, freckled skin, cutaneous pigmented spot, a problem with an eyelid, skin topography, a sensitive lip, wrinkles around a lip, acne, eczema, dandruff or any other skin condition. Keratin characteristics and conditions may relate to at least one of hair, nails, eyelashes, and eyebrows. Keratin conditions may include hair loss, hair shine, hair thickness, hair oiliness, hair health, hair graying, hair color, dirty hair, curly hair, straight hair, limp hair, or any other keratin condition.

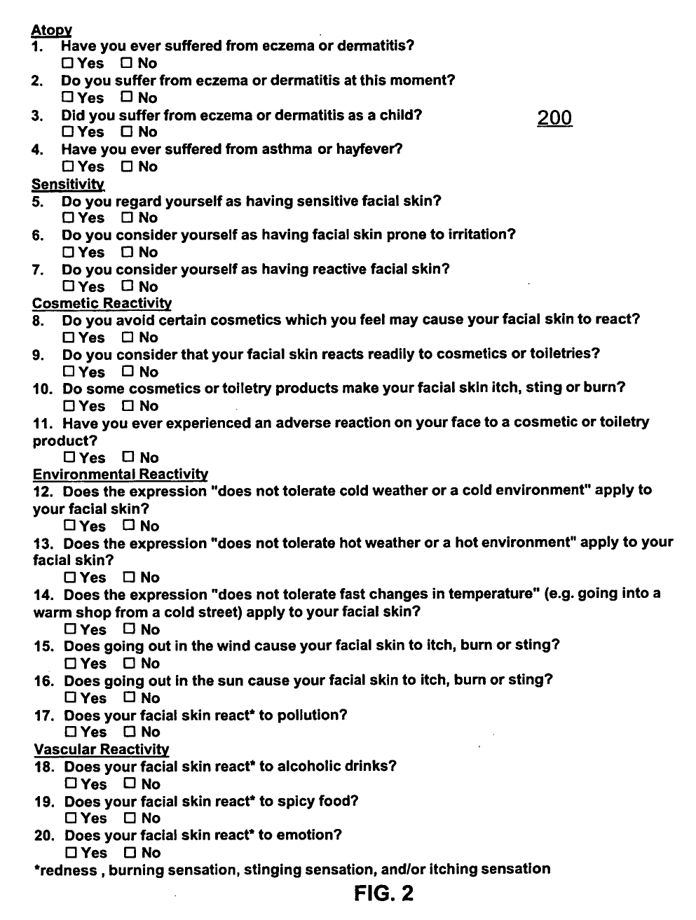

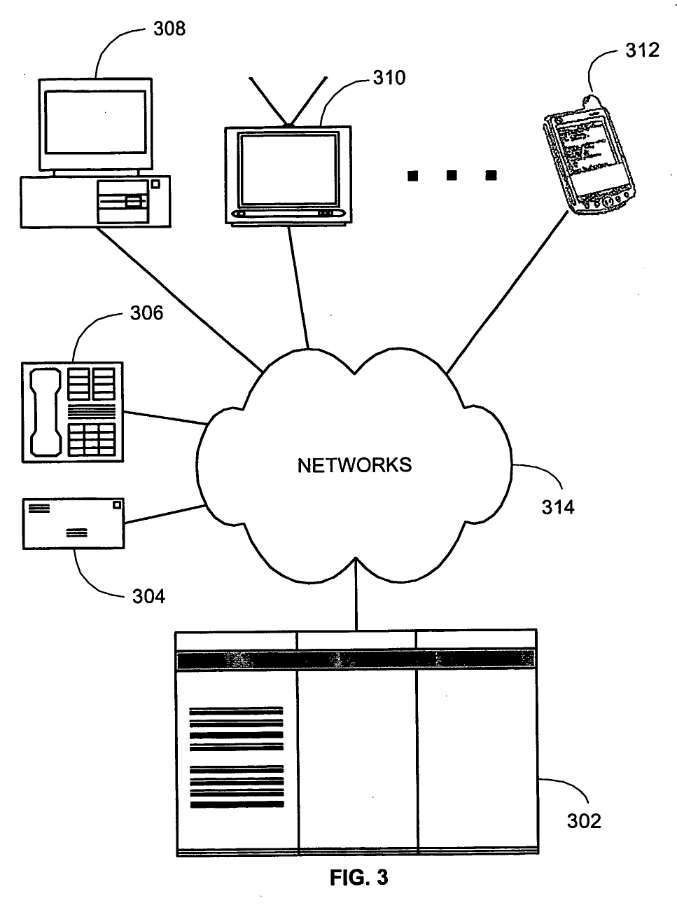

For example, questionnaire 200 in FIG. 2 illustrates a number of questions relating to various sub-topics. In particular, questions 1-4, 5-7, 8-11, 12-17, and 18-20 relate to atopy, self-perceived sensitivity, cosmetic reactivity, environmental reactivity, and vascular reactivity, respectively. As illustrated in FIG. 3, questionnaire 200 may be presented to a plurality of individuals by computer system 302 via paper, magazine, leaflet 304, telephone 306, computer 308, television 310, personal electronic organizer 312, and any other suitable mechanism over one or more networks 314.

According to features and principles consistent with the present invention, method 100 may further include receiving information reflecting answers to the questions (step 104 in FIG. 1). “Receiving” may include receiving the information over a network or via an electronic device. Alternatively or additionally, “receiving” may include receiving the information from a database, a data structure, a storage medium, and/or any other mechanism or combination of mechanisms. The information may reflect answers to questions from a survey, such as questionnaire 200 in FIG. 2. The answers may have one or more modalities. Each modality may correspond to a particular answer to the question. For example, each question in questionnaire 200 has two answers (i.e., two modalities). The individuals may answer the questions by selecting one of the two modalities for each question. Their answers may then be stored in a database that is made available for use by computer system 302 in FIG. 3.

Consistent with features and principles of the present invention, receiving the information reflecting the plurality of individuals' answers to the questions may also include collecting answers to the questions.

According to features and principles consistent with the present invention, method 100 may further include performing an analysis on the received information to generate a synthetic variable (step 106). The analysis may be performed by a data processor, such as a computer system, a microchip, or any other form of processor. For example, computer system 302 may contain instructions and/or software stored in a storage medium (e.g., memory, electromagnetic storage device, computer program product, computer-readable medium, etc.). The instructions and/or software may configure computer system 302 to perform the analysis on the received information to generate the synthetic variable. Alternatively or additionally, the analysis may be performed at least in part by a researcher, analyst, statistician, or any other type of expert or facilitator. The analysis may include a multivariate analysis, which may include at least one of a principal component analysis, a factorial analysis, PLS path modeling, structural equation modeling, and any other type of mathematical analysis.

The analysis may be hierarchical whereby the synthetic variable is generated by consecutive multivariate analyses, each multivariate analysis being performed on the results of a previous multivariate analysis. For example, a hierarchical principal component analysis may generate output variables using a principal component analysis on intermediate variables from another principal component analysis.

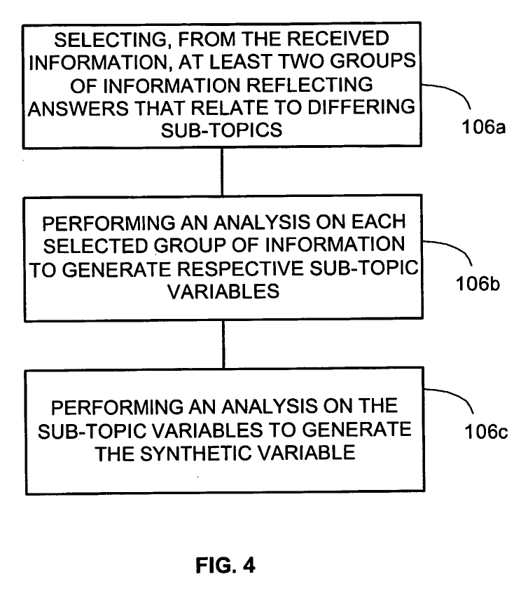

Consistent with features and principles of the present invention, performing the analysis may comprise selecting, from the received information, at least two groups of information reflecting answers that relate to differing sub-topics, respectively, as illustrated in FIG. 4 (step 106a). The differing sub-topics may include one or more of the sub-topics listed above. “Selecting” may include identifying answers of questions previously associated with a sub-topic. For example, the answers, from a plurality of individuals, to questions 1-4 in FIG. 2 may have been previously associated with atopy and computer system 302 may select a group of information reflecting the answers to questions 1-4. “Selecting” may include identifying answers to questions associated in real time with a sub-topic. For example, computer system 302 may evaluate the received information from step 104 and associate questions 8-11 in FIG. 2 with cosmetic reactivity in real time.

Consistent with features and principles of the present invention, performing the analysis may further include performing an analysis on each selected group of information to generate respective sub-topic variables, as illustrated in FIG. 4 (step 106b). The analysis on each selected group of information may use any of the multivariate analyses listed above and/or described in A User's Guide to Principal Components, J. Edward Jackson, John Wiley & Sons, Inc., New York, 1991, which is incorporated herein by reference. The analysis may also be performed using software tools, such as SPSS 11.0 from SPSS, Inc. located at 233 S. Wacker Drive, 11th floor, Chicago, Ill. 60606; SAS 8.2 from SAS Institute Inc located at SAS Campus Drive Cary, N.C. USA; S-PLUS from Mathsoft International located at Knightway House Park Street Bagshot, Surrey UK; SPAD from DECISIA located at 261 rue de Paris 93556 Montreuil cedex, France, etc. For example, the analysis may be a principal component analysis and each sub-topic variable may be from the first factorial axis of the principal component analysis for each sub-topic's group of information.

More specifically, a principal component analysis on the answers to questions 1-4 for atopy in questionnaire 200 may yield one or more factorial axes. Assuming the answers to questions 1-4 have the largest variance along the first factorial axis, a sub-topic score/value for each individual may be calculated from the first principal component of a principal component analysis on questions 1-4. The principal component may be viewed for each individual as a projection of the vector of answers to questions 1-4 onto the first factorial axis. The sub-topic variable for atopy may be formed from the atopy sub-topic scores/values of all the individuals. Similarly, sub-topic variables relating to sensitivity, cosmetic reactivity, environmental reactivity, and vascular reactivity, may be generated using answers to questions 5-7, 8-11, 12-17, and 18-20, respectively. 0

Consistent with features and principles of the present invention, performing the analysis may further include performing an analysis on the sub-topic variables to generate the synthetic variable, as illustrated in FIG. 4 (step 106c). The analysis on the sub-topic variables may use any of the multivariate analyses listed above. The synthetic variable may be from the first factorial axis of the principal component analysis of two or more sub-topic variables. The analysis may be a principal component analysis performed in a manner similar to the one discussed above for the sub-topic variables in step 106b.

According to features and principles consistent with the present invention, method 100 may further include transforming the synthetic variable with a linear transformation, as shown in FIG. 1 (step 108). “Transforming” may include performing one or more mathematical operations on the synthetic variable to change the synthetic variable into a score from 0 to 10 or any other interpretation of the synthetic variable.

According to features and principles consistent with the present invention, method 100 may further include generating a diagnostic algorithm from at least the synthetic variable using a tree segmentation technique, as illustrated in FIG. 1 (step 110). Generating the diagnostic algorithm may include selecting most discriminating questions and generating a limited set of the most discriminating questions that can provide a diagnosis by progressing through a tree.

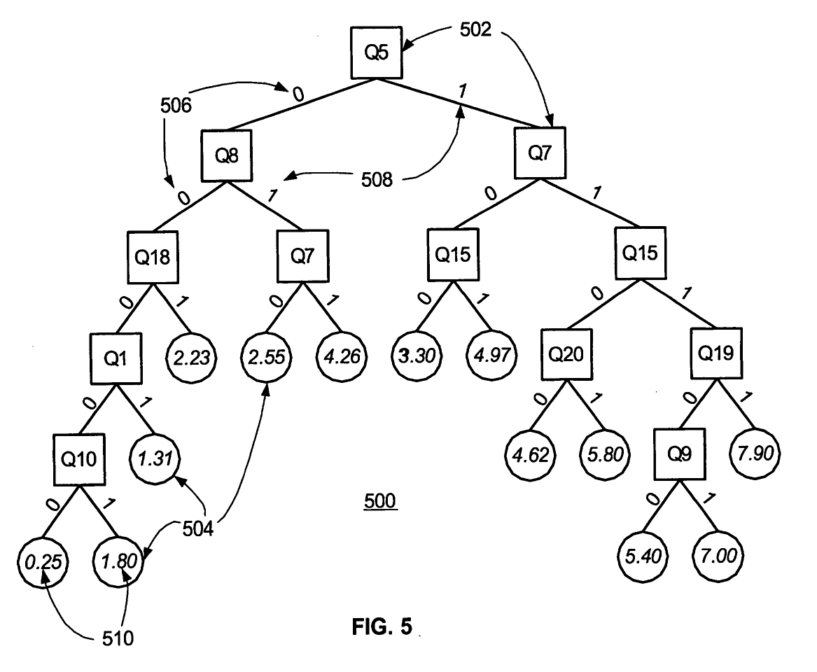

The diagnostic algorithm may be a branching tree of discriminating questions or any other type of diagnostic algorithm based on the synthetic variable. For example, the diagnostic algorithm may be the binary tree 500 illustrated in FIG. 5. Binary tree 500 may include decision points/branches 502 (i.e., squares) and termination points/branches 504 (i.e., circles). Each decision point 502 may correspond to a discriminating question in questionnaire 200 of FIG. 2 with modality zero 506 for a “No” answer and modality one 508 for a “Yes” answer. Each termination point 504 may correspond to a synthetic variable value or a score 510 that provides an indication of a diagnosis for any subject who progresses through binary tree 500, answers discriminating questions at decision points 502, and terminates at one of the termination points 504. The synthetic variable value or score 510 associated with the subject's termination point may be the diagnosis for the subject.

The diagnostic algorithm may be generated using a tree segmentation technique, such as a classification and regression tree method or any method described in The Elements of Statistical Learning, Trevor Hastie et al., Springer-Verlag New York, Inc., New York, 2001, and/or Classification and Regression Trees, Leo Breiman, Wadsworth, Inc., California, 1984. The diagnostic algorithm may also be generated, at least in part, with the help of software tools, such as the software tools previously discussed. The classification and regression tree method may include a CART method, a CHAID method, a QUEST method, or another appropriate mathematical method. For example, as one of ordinary skill in the art will appreciate, computer system 302 may use the CART method to compute a recursive binary partition of the answers to the questions in questionnaire 200 (FIG. 2) with the synthetic variable as a response variable in the CART method.

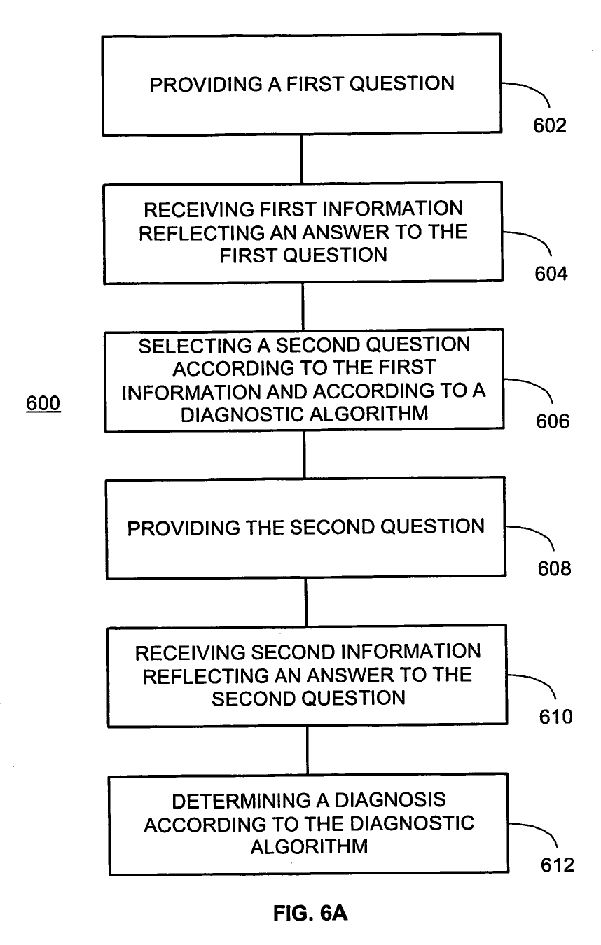

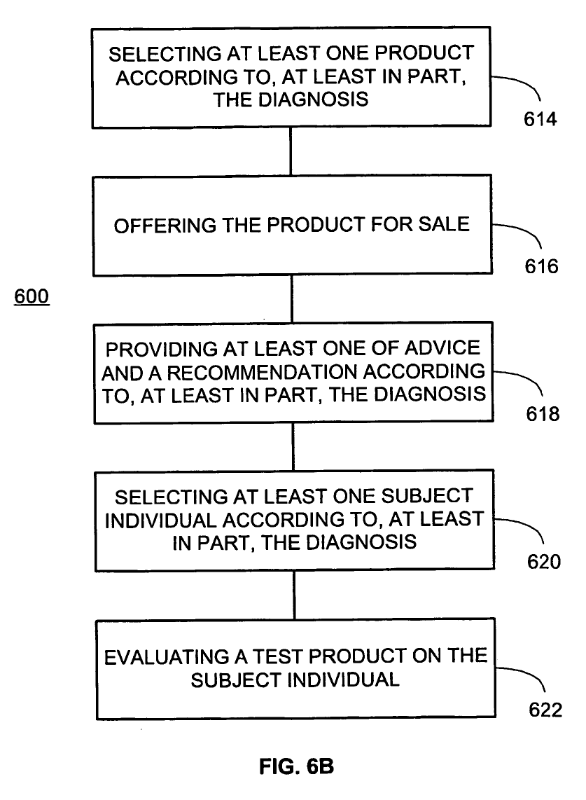

Features and principles consistent with the present invention may include a diagnostic method 600, as illustrated in FIGS. 6A and 6B. Diagnostic method 600 may be implemented by an electronic processing device or a combination of the electronic processing devices described above. Diagnostic method 600 may reduce the time necessary for the electronic device to diagnose a subject. In turn, this may increase a battery life or power efficiency of the electronic device. Diagnostic method 600 may also lower the memory storage requirements for the electronic device by reducing the amount of memory required to generate a diagnosis using the device.

According to features and principles consistent with the present invention, diagnostic method 600 may comprise providing a first question (step 602). “Providing” the first question may include providing the first question in a manner previously described above for “presenting.” The first question may be a most discriminating question, such as question Q5 at the top of binary tree 500 in FIG. 5. Diagnostic method 600 may further include receiving first information reflecting an answer to the first question (step 604). “Information” as used herein may be data in any form or medium.

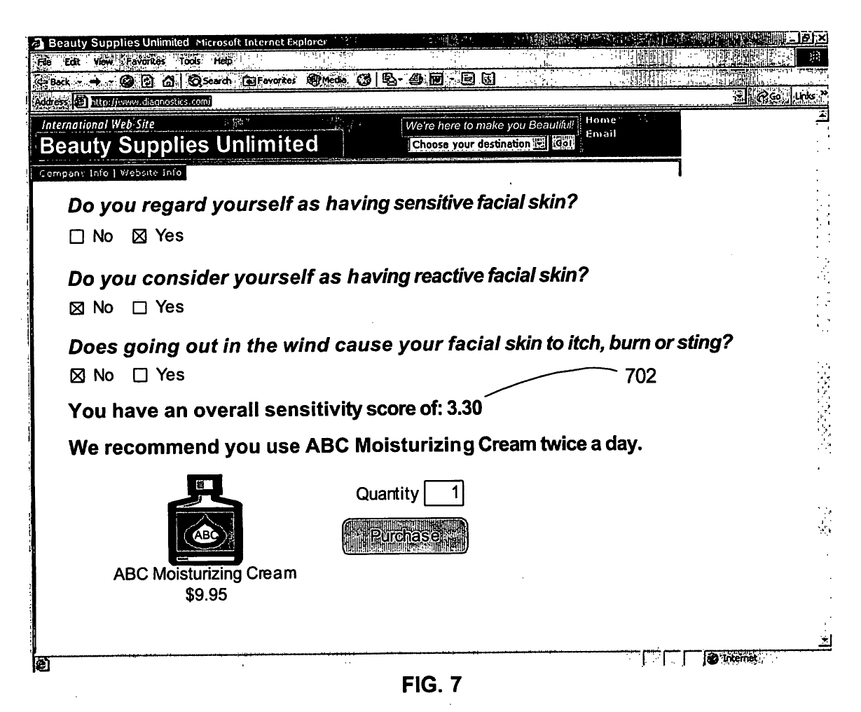

For example, a computer server may provide question Q5 to a subject via a wireless internet connection to the subject's personal electronic organizer (e.g., a personal digital assistant). On the display, the subject may see a small web page displaying the words, “Do you regard yourself as having sensitive facial skin?,” as illustrated in FIG. 7. The subject may respond by indicating that she considers herself as having sensitive facial skin and the computer server may receive information reflecting her response.

According to features and principles consistent with the present invention, diagnostic method 600 may further include selecting a second question according to the first information and a diagnostic algorithm generated using at least one of a multivariate analysis and a tree segmentation technique (step 606). The diagnostic algorithm may be one similar to binary tree 500 in FIG. 5. The second question may be, according to the tree segmentation technique and the answer to the first question, a second most discriminating question. Continuing the above example illustrated in FIG. 5, since the subject's answer to the first question Q5 was a “yes,” the computer server may select the next question to be question Q7, which is the second most discriminating question according to binary tree 500 and the subject's answer to the first question.

According to features and principles consistent with the present invention, diagnostic method 600 shown in FIG. 6A may further include providing the second question (step 608), and receiving second information reflecting an answer to the second question (step 610). Hence, from the above example illustrated in FIG. 5, the computer server may provide question Q7 in binary tree 500 to the subject. As shown in FIG. 7, the subject may, on the display of her personal electronic organizer, see the words, “Do you consider yourself as having reactive facial skin?” The subject may respond with a “no” and the computer server may receive information reflecting her response.

According to features and principles consistent with the present invention, diagnostic method 600 shown in FIG. 6A may further include determining a diagnosis according to the diagnostic algorithm (step 612). In the above example, since the subject's negative response to question Q7 does not take her to a termination point 504 in binary tree 500, diagnostic method 600 may further include selecting at least one subsequent question according to the diagnostic algorithm, providing the at least one subsequent question, and/or receiving subsequent information reflecting at least one answer to the at least one subsequent question until a diagnosis is determined. For example, the subject's next question in binary tree 500 is question Q15. Depending on whether she answers question Q15 with a negative or affirmative, the computer server may, according to binary tree 500, determine a diagnosis of her overall sensitivity score to be 3.30 or 4.97, respectively, and provide her with the diagnosis 702, as shown in FIG. 7. Note that in this example the subject only answered three questions, instead of a possible twenty questions shown in FIG. 2, to obtain the diagnosis.

According to features and principles consistent with the present invention, diagnostic method 600 may further include selecting at least one product according to, at least in part, the diagnosis, as illustrated in FIG. 6B (step 614). The product may be particularly suited to individuals with similar diagnoses or the same diagnosis as the subject. A “product” as used herein may include any tangible merchandise, good, service, and/or action performed. Examples of tangible merchandise forms of products may include cosmetic goods, such as treatment products, personal cleansing products, and makeup products, in any form (e.g., ointments, creams, gels, sprays, supplement, ingesta, inhalants, lotions, cakes, liquids, and powders). Examples of service forms of products may include hair styling, hair cutting, hair coloring, hair removal, skin treatment, make-up application, and any other offering for aesthetic enhancement. Examples of other actions performed include massages, facial rubs, deep cleansings, applications of beauty product, exercise, therapy, or any other action affecting the body or face whether performed by a professional, the subject, or an acquaintance of the subject.

The following is exemplary and non-exhaustive listing of a few products: scrubs, rinses, washes, moisturizers, wrinkle removers, exfoliates, toners, cleansers, conditioners, shampoos, cuticle creams, oils, anti-fungal substances, anti-aging products, anti-wrinkle products, anti-freckle products, skin conditioners, skin toners, skin coloring agents, tanners, bronzers, skin lighteners, hair coloring, hair cleansing, hair styling, elasticity enhancing products, agents, blushes, mascaras, eyeliners, lip liners, lipsticks, lip glosses, eyebrow liners, eye shadows, nail polishes, foundations, concealers, dental whitening products, cellulite reduction products, hair straighteners and curlers, and weight reduction products.

According to features and principles consistent with the present invention, diagnostic method 600 may further include offering the product for sale (step 616). “Offering” the product may include supplying any information to the subject that will facilitate the subject or a third party in purchasing the product. In the above example, the computer server may send information such as the product's price and quantity available to the subject.

According to features and principles consistent with the present invention, diagnostic method 600 may additionally or as an alternative to steps 614 and 616 include providing at least one of advice and a recommendation according to, at least in part, the diagnosis (step 618). “Advice” may include any provision of information related to the diagnosis. A “recommendation” may include one or more product recommendations (e.g., cosmetic product recommendations for products to treat diagnosed conditions of the subject), remedial measures, preventative measures, predictions, prognoses, price and availability information, application and use information, suggestions for complementary products, lifestyle or dietary recommendations, or any other information intended to aid the subject in a course of future conduct, to aid the subject in understanding past occurrences, to reflect information about some future occurrences related to the subject or to aid the subject in understanding products, as defined above.

According to features and principles consistent with the present invention, diagnostic method 600 may additionally or as an alternative to steps 614-618 include selecting at least one subject individual according to at least in part the diagnosis (step 620) and/or evaluating a test product on the subject individual (step 622). The diagnosis may indicate that the subject has or exhibits a characteristic or quality that is useful for certain types of tests. For example, the subject may have sensitive skin and a formulator, manufacturer, distributor, etc. may wish to test whether a test product, such as experimental make-up, generally affects individuals with sensitive skin. The test product may include any product described above.

Features and principles consistent with the present invention may include a diagnostic method 800, as illustrated in FIG. 8. Diagnostic method 800 may comprise receiving a first question (step 802) and sending first information reflecting an answer to the first question (step 804). “Sending” the first information may include sending the information over a network, via an electronic device, or in any other manner. Alternatively or additionally, “sending” may include sending the information to a database, a data structure, a storage medium, and/or any other mechanism or combination of mechanisms. “Sending” may occur through an on-line interest form, e-mail, facsimile, telephone, interactive voice response system, or file transfer protocol transmitted electronically over a network at a web site, an internet protocol address, or a network account. The sent information may take one of many forms. It may, for example, be a clicked button, submitted form, or oral affirmation. It might be typed or handwritten text. In the example illustrated in FIG. 7, the sent information may be a checked box.

According to features and principles consistent with the present invention, diagnostic method 800 may further include receiving a second question, wherein the second question is a question selected according to the first information and according to a diagnostic algorithm generated using at least one of a multivariate analysis and a tree segmentation technique (step 806), and sending second information reflecting an answer to the second question, wherein a diagnosis is determined according to the diagnostic algorithm (step 808). The diagnostic method 800 may further include receiving third information reflecting the diagnosis (step 810). The third information may include quantitative information (e.g., a score, a numeric rating, etc.) or qualitative information (e.g., a description, a category, a type, etc.) expressing the diagnosis.

According to features and principles consistent with the present invention, diagnostic method 800 may further include selecting at least one product according to, at least in part, the diagnosis (step 812). For example, a subject may select a certain beauty product according to the results of the diagnosis. Diagnostic method 800 may, additionally or as an alternative to step 812, include receiving at least one of advice and a recommendation according to, at least in part, the diagnosis (step 814). For example, the subject may receive a recommendation for and advice to use ABC moisturizing cream, as illustrated in FIG. 7.

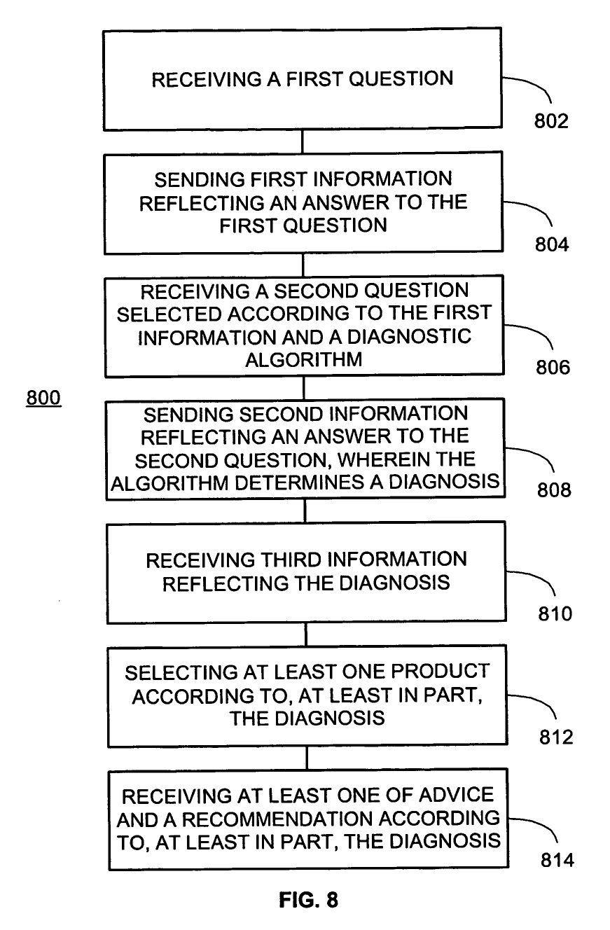

In one exemplary embodiment consistent with features and principles of the present invention, a research company sent questionnaire 200 (FIG. 2) to 5000 women. The company received 1206 responses, of which 1037 had no missing data. FIG. 9 displays the incidence of positive responses for each question in the questionnaire.

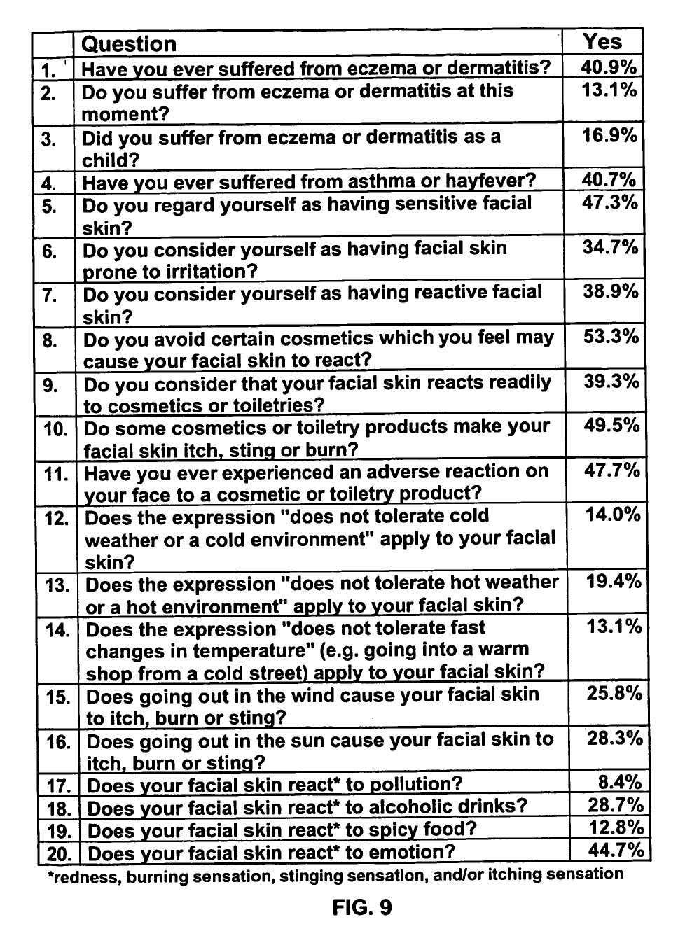

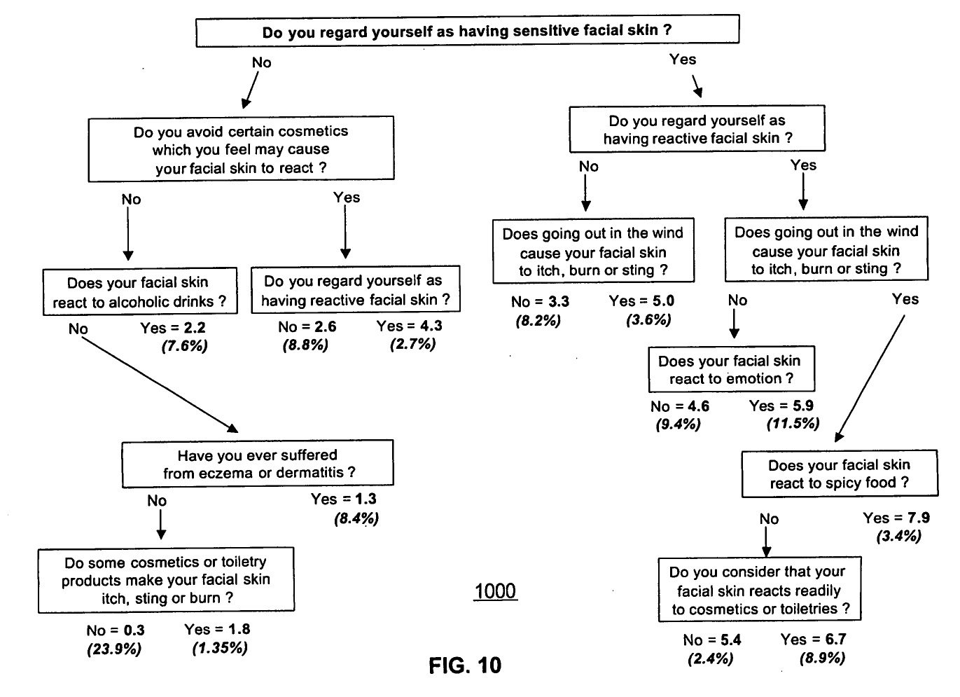

From the 1037 responses, diagnostic algorithm 1000 in FIG. 10 was generated using methods consistent with features and principles of the present invention. The numbers after the Yes/No answers are the diagnostic scores and the numbers in parentheses under the Yes/No answers indicate the percentage of the 1037 people grouped with the same diagnostic score. Note the first question in diagnostic algorithm 1000 is question 5 (Q5), “Do you regard yourself as having sensitive facial skin?”

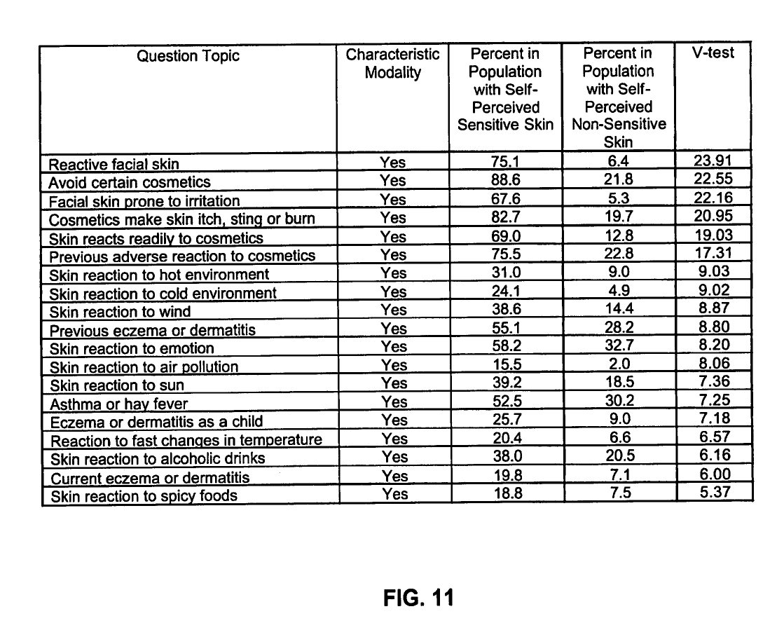

FIG. 11 displays comparisons made between a self-perceived, sensitive skin subpopulation (i.e., those who responded “yes” to question 5 and are on the right side of FIG. 10) and a self-perceived, non-sensitive skin subpopulation (i.e., those who responded “no” to question 5 and are on the left side of FIG. 10) with respect to the other 19 questions in questionnaire 200. FIG. 9 arranges the other 19 questions in descending order of statistical significance, as determined by the Value test (V-test). Variables with V-tests of greater than 2.0 were judged statistically significant at the 5% level (reference: Ludovic Lebart, Alain Morineau, Marie Piron: Statistique exploratoire multidimensionnelle, dunod 1995). The modality “yes” was significantly overexpressed in the self-perceived sensitive skin subpopulation for all the other 19 questions. The major discrepancies between the two subpopulations concerned self-perceived reactive and prone-to-irritation skin (questions 6 and 7 in FIG. 2). To a lesser extent, the self-perceived sensitive skin subpopulation presented a higher incidence of atopy, cosmetic reactivity, environmental reactivity, and vascular reactivity (questions 1-4 and 8-20).

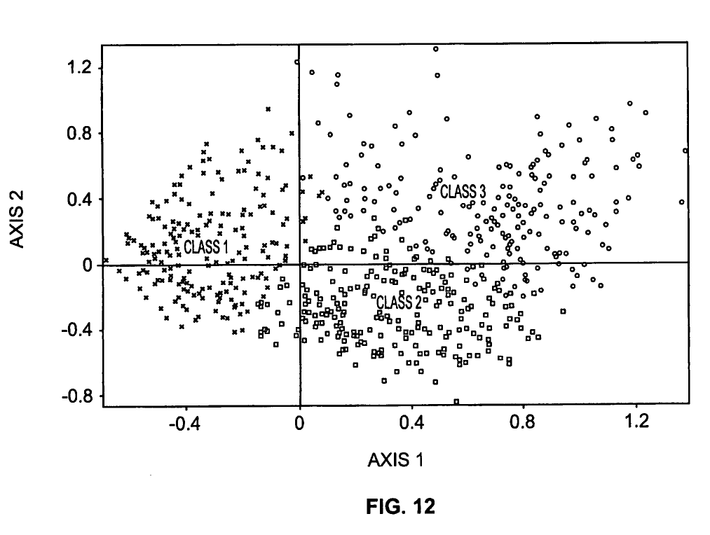

FIG. 12 displays a typology constructed by factorial analysis on the 1037 responses. An automatic hierarchical ascending classification was performed to create classes gathering individuals with homogeneous profiles of response to questionnaire 200. This non-supervised factorial data analysis proposed the three-class distribution illustrated in FIG. 12, which displays a projection of the individuals in a first factorial plane according to a symbol code proper to each class.

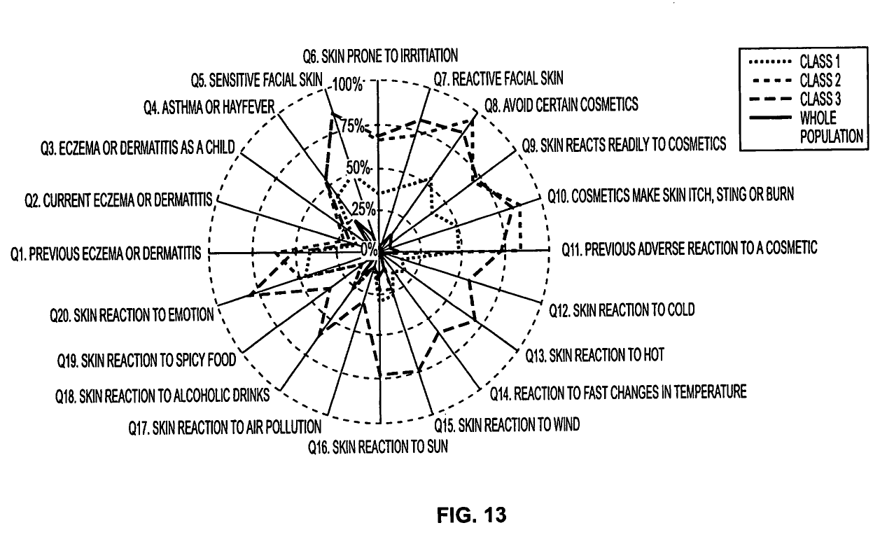

FIG. 13 shows an average profile of positive responses to the three different classes. The first class gathered individuals with non-sensitive facial skin and had a population of 514 individuals with a mean age of 49.6 years. The second class included individuals with self-perceived sensitive facial skin characterized by a high incidence of cosmetic reactivity (i.e., high rates of positive responses to questions 5-11) and had a population of 329 individuals with a mean age of 42.2 years. The third class brought together individuals with self-perceived sensitive facial skin characterized by a high incidence of both cosmetic reactivity and environmental reactivity (i.e., high rates of positive responses to questions 5-17) and had a population of 194 individuals with a mean age of 45.4 years. Individuals in the third class also reported more frequent incidences of vascular reactivity. The third class was more distant from the first class on the first factorial plane (FIG. 12) and presented a greater number of triggering factors than the second class. This suggested that the third class corresponded globally to a more severe condition of sensitive skin than the second class.

As generally indicated by the above results, questionnaire 200 appeared to be a good tool to assess the incidence of self-perceived sensitive skin. The incidence of self-perceived sensitive skin was found to be approximately 50%. A stronger skin reactivity to cosmetics appeared as the main characteristic of sensitive skin. Skin reactivity to environmental factors was, for a large part, associated with reactivity to cosmetics and linked to self-perceived sensitive skin.

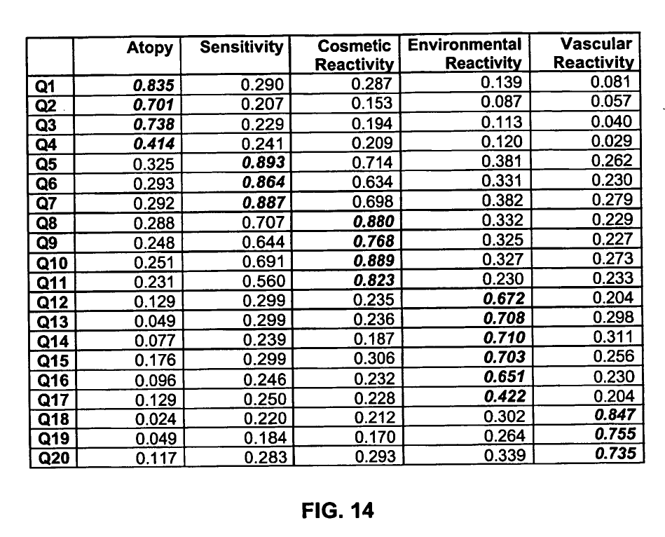

FIG. 14 illustrates the correlations between questions 1-20 and the sub-topic variables. Overall the sub-topic variables had a strong correlation with their associated questions and a weak correlation with unassociated questions. Questions 4 and 17 had correlation coefficients of 0.414 and 0.422 with their respective sub-topic variables, and appeared weakly correlated with them. However, they remained more strongly correlated with their sub-topic variables than they did with other sub-topic variables.

Questions 5 and 7 of the self-perceived sensitive sub-topic appeared to be correlated with the cosmetic reactivity sub-topic, as illustrated in FIG. 14. Conversely, questions 8 and 10 of the cosmetic sub-topic seemed to be correlated with the self-perceived sensitive sub-topic even if these associations were less than their association with the cosmetic reactivity sub-topic to which they belonged.

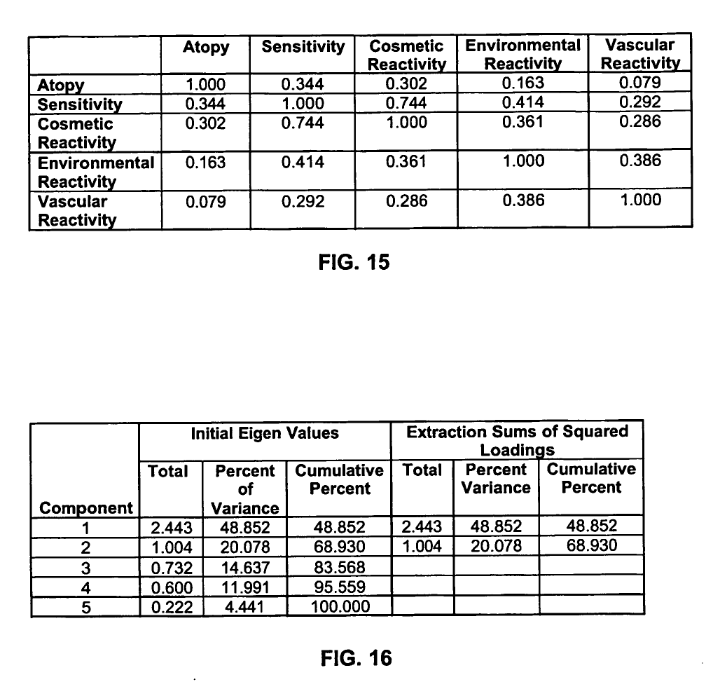

FIG. 15 presents the correlations between the various sub-topics. Weak correlations were observed between the sub-topics, except for the self-perceived sensitivity and cosmetic reactivity sub-topics, which seemed to be related with a coefficient of 0.744.

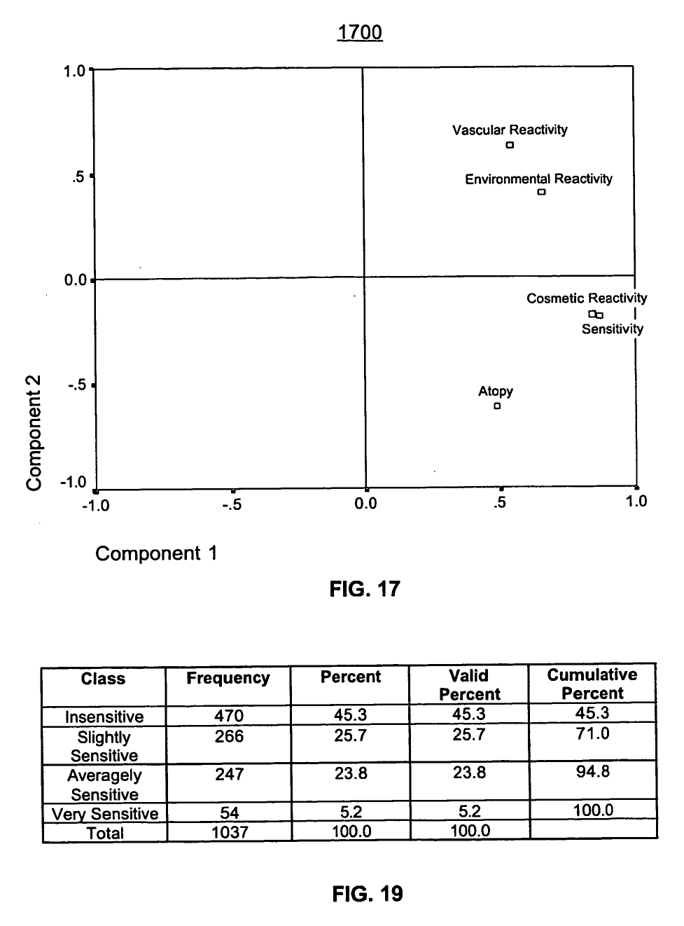

The principal component analysis of the five sub-topics retained only two factors. The first factorial plane explained 68.93% of the total variance, as shown in FIG. 16. Graph 1700 in FIG. 17 presents the projection of the sub-topics onto the first factorial plane. The horizontal axis is a overall sensitivity axis, where the most sensitive individuals lie to the far right in graph 1700. The vertical axes separates the atopic individuals from the environmentally and/or vascularly reactive individuals. By choosing three classes, a typology (k-means) was constructed around the coordinates of the factorial axes of the principal component analysis.

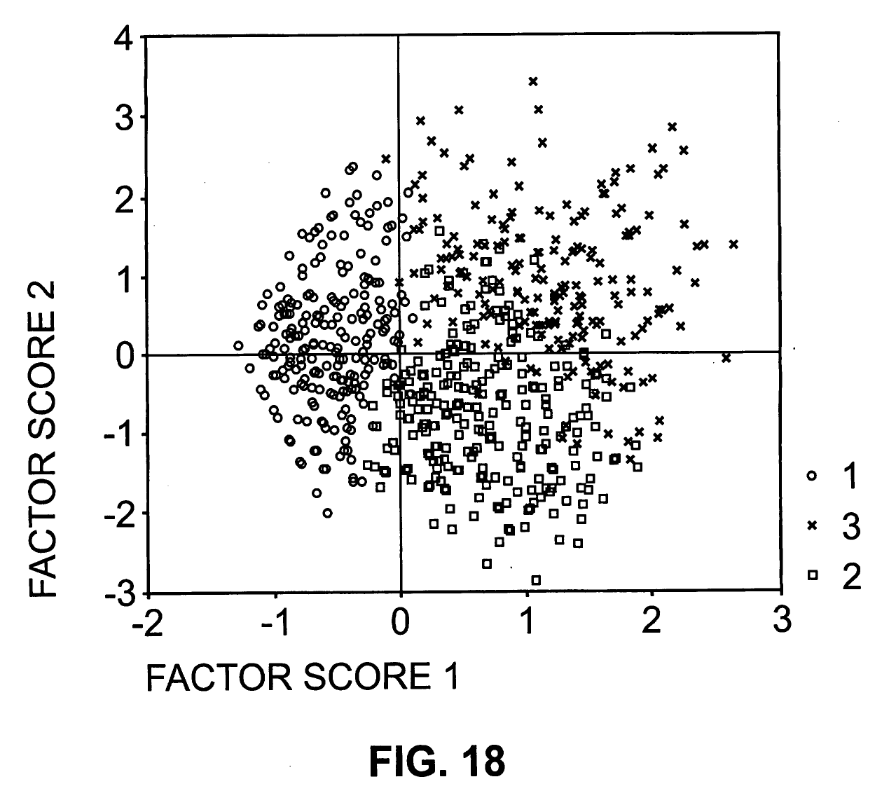

FIG. 18 presents the projection of the 1037 individuals onto the first factorial plane. Since the first factorial axis appeared to be an overall sensitivity axis, the individuals were projected onto the factorial axis. Further, the first factorial axis was sliced into four equal length intervals that were associated with insensitive, slightly sensitive, moderately sensitive, and very sensitive classes. The distribution of the individuals as a function of these sensitivity classes is presented in FIG. 19.

A diagnostic algorithm based on the questions in questionnaire 200 was then constructed in order to provide a qualitative diagnosis for any given subject by assigning her to one of the classes. A tree-based segmentation technique (e.g., the CART method) was used. At each step, the CART method operated by dichotomy of a selected predictor, which in this case was a question in questionnaire 200. The CART method split each segment into two sub-segments such that each “child” segment was purer than the “parent” segment. The concept of purity refers to the distribution of individuals in the sensitivity classes. A perfectly pure segment corresponds to a situation in which all the individuals belongs to the same overall sensitivity class.

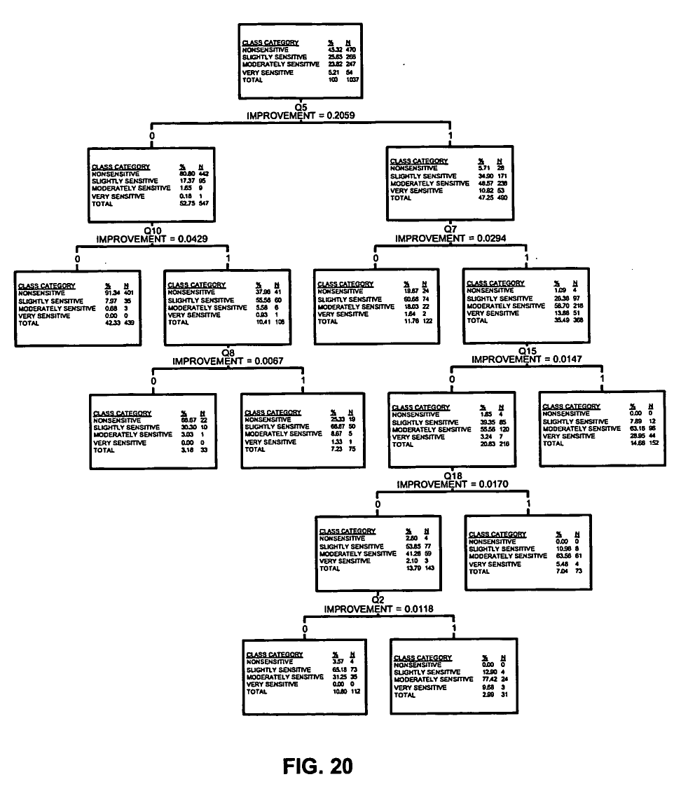

The tree in FIG. 20 presents the diagnostic algorithm for the distribution into four classes. The initial segment corresponded to the entire set of individuals. The most discriminating question was question 5 (Q5), which segmented the population into two more homogeneous sub-populations. As shown in FIG. 20, 80.80% of the individuals who responded “no” to question 5 (i.e., Q5=0) were insensitive, whereas only 5.71% of the individuals who responded “yes” (i.e., Q5=1) were insensitive.

Accordingly, more homogeneous segments were obtained. Particularly, in the next step, for individuals who responded Q5=0, the most discriminating question was question 10 (Q10), which separated the insensitive individuals from the slightly sensitive individuals. 91.34% of the individuals who responded Q5=0 and Q10=0 were associated with the insensitive class. This segment was deemed a final segment and was not broken down further. For the individuals who responded Q5=1, the most discriminating question was question 7 (Q7). Question 7 created two segments, one with 60.66% insensitive individuals, which was a final segment, and the other with 58.70% moderately sensitive individuals, which was broken down further. Segmentation was continued until the final segments were characterized by the majority sensitivity class. A simple rule of assignment to one of the four sensitivity classes was thus obtained for any subject individual on the basis of her response to the discriminating questions indicated in FIG. 20.

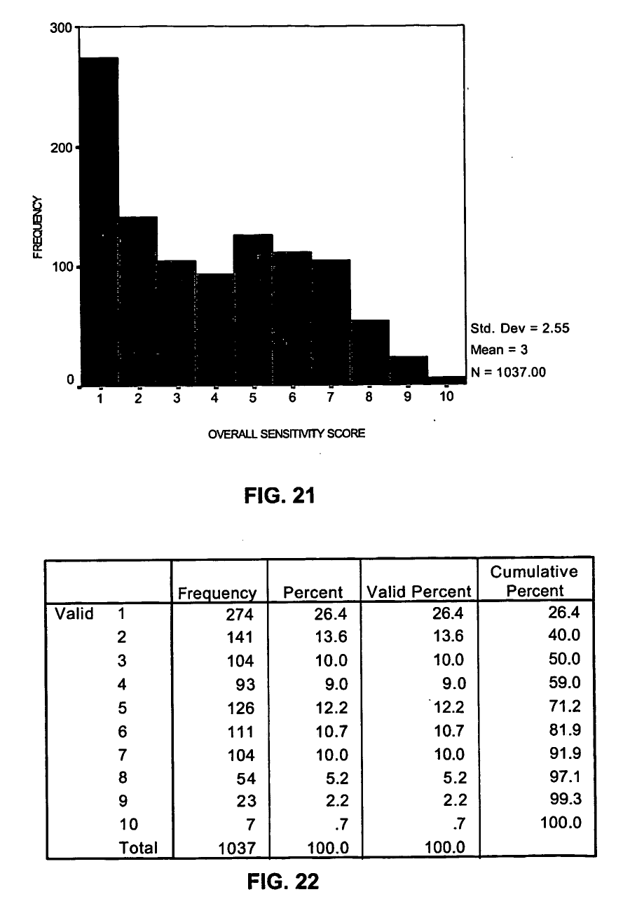

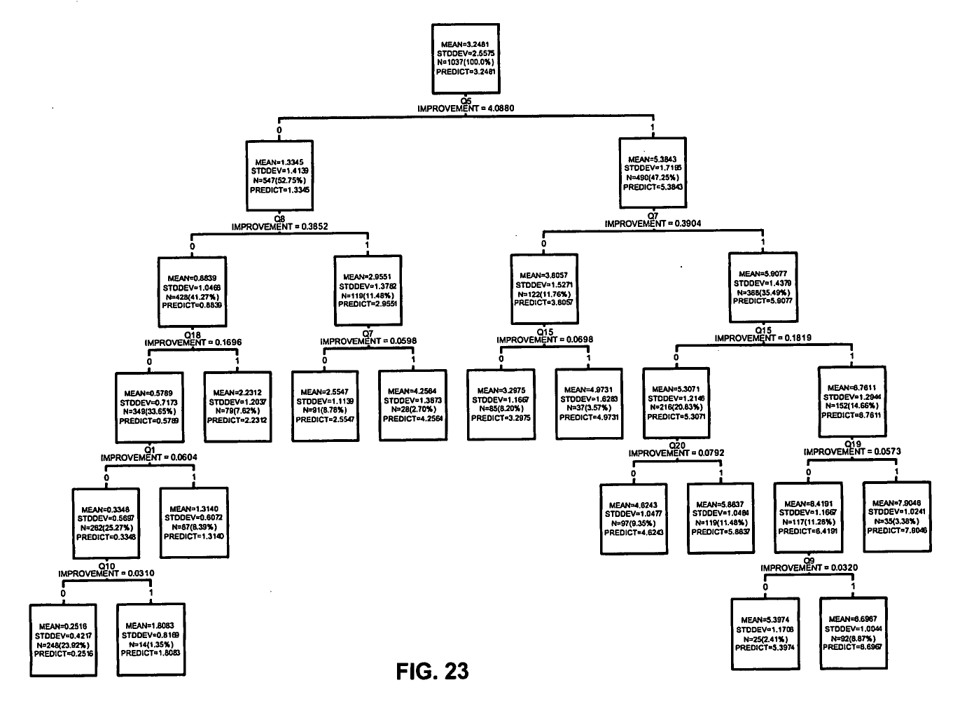

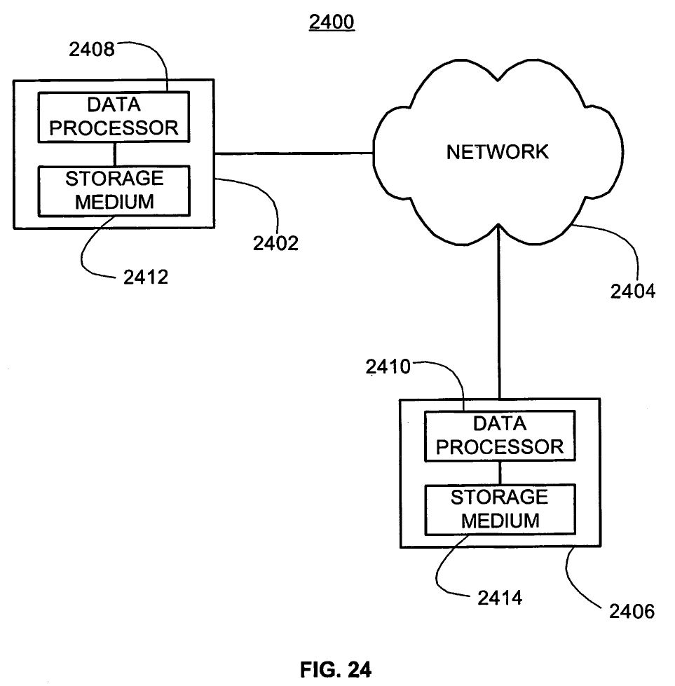

In another exemplary embodiment consistent with features and principles of the present invention, a diagnostic algorithm was constructed in order to assign an overall sensitivity score to a subject. Instead of using the CART method to split segments to obtain purer segments for a class as discussed above for the diagnostic algorithm in FIG. 20, the CART method was used on a synthetic variable generated from the projection of the individuals onto the first factorial axis of the principal component analysis. The synthetic variable was then transformed into a overall sensitivity score varying from 0 to 10. FIGS. 21 and 22 illustrate the histogram of the scores and lists their frequency, respectively. FIG. 23 shows the diagnostic algorithm generated from the CART method. Question 5 separates the population into two sub-populations of mean score 1.3 for Q5=0 and 5.4 for Q5=1. Subsequent questions separate the sub-populations up to termination points determined by the CART method.

Although some of the above embodiments describe using features and principles of the present invention to diagnose a skin sensitivity score, other synthetic variables may also be used. For example, a list of questions designed to evaluate an intelligence quotient (IQ) of an individual may be posed to a sample population of individuals. Using the analysis/analyses and techniques described above, an algorithm may be designed to efficiently determine the IQ of a subject. The algorithm would ask the subject a limited number of questions as it progressed through a tree, until it terminated at a terminating branch. An IQ score associated with the terminating branch would then be applied to the subject.

Some other topics/themes which can be explored and characterized by a synthetic variable may include: greasy skin, dry skin, hair loss, aging skin, wrinkled skin, marked skin, flask skin, squeamish skin, sun-sensitive skin, profile of a cosmetic products consumer, “bags under the eyes”, the most suitable make-up, problem with the contour of the eyes, topographic analysis of the face including the neck (for example, one or more zones especially affected by aging), and neckline (for example, one or more zones especially affected by photo-aging, photo-damaging), hair greying (for example, in the context of capillary dyes/colorant use), the perioral zone (e.g., sensitive lips or wrinkles around the lips), acne, eczema, problems affecting the entire body (e.g., cellulite), and questions like “are your cosmetic habits well-adapted to your skin type or your general state”. There is no limitation in the kind of theme/topic which could be explored. They may only depend on a database generated from answers collected in relevant questionnaires.

Features and principles consistent with the present invention may include diagnostic system 2400 illustrated in FIG. 24. Diagnostic system 2400 may include a first computer 2402, a network 2404, and a second computer 2406. Network 2404 may include any network previously discussed. Computer 2402 may include a data processor 2408 and a storage medium 2412. Computer may include a data processor 2410 and a storage medium 2414. Data processors 2408 and 2410 and storage mediums 2412 and 2414 may respectively include any data processor and storage medium previously discussed.

Computer 2402 may be configured to implement diagnostic method 600 in FIGS. 6A and 6B. For example, storage medium 2412 may contain instructions configuring data processor 2408 to implement method 600.

Computer 2406 may be configured to implement diagnostic method 800 in FIG. 8. For example, storage medium 2414 may contain instructions configuring data processor 2410 to implement method 800.

In the foregoing description, various features are grouped together in various embodiments for purposes of streamlining the disclosure. This method of disclosure is not to be interpreted as reflecting an intention that the claimed invention requires more features than are expressly recited in each claim. Rather, as the following claims reflect, inventive aspects may lie in less than all features of a particular embodiment described above. Thus, the following claims are hereby incorporated into this description.

What is claimed is:

1. A diagnostic method comprising:
providing a first question;
receiving first information reflecting an answer to the first question;
selecting a second question according to the first information and according to a diagnostic algorithm generated using at least one of a multivariate analysis and a tree segmentation technique;
providing the second question;
receiving second information reflecting an answer to the second question; and
determining a diagnosis according to the diagnostic algorithm.

2. The method according to claim 1, wherein the multivariate analysis includes at least one of a principal component analysis, a factorial analysis, PLS path modeling, and structural equation modeling.

3. The method according to claim 1, wherein the tree segmentation technique includes a classification and regression tree method.

4. The method according to claim 3, wherein the classification and regression tree method is at least one of a CART method, a CHAID method, and a QUEST method.

5. The method according to claim 1, wherein the diagnostic algorithm is a diagnostic algorithm generated using at least both the multivariate analysis and the tree segmentation technique.

6. The method according to claim 5, wherein the multivariate analysis includes a principal component analysis and wherein the tree segmentation technique includes a classification and regression tree method.

7. The method according to claim 1, wherein the first question is a most discriminating question according to the tree segmentation technique.

8. The method according to claim 7, wherein the second question is, according to the tree segmentation technique and the answer to the first question, a second most discriminating question.

9. The method according to claim 1, wherein at least one of the first information and the second information is received from a remote location over a network.

10. The method according to claim 9, wherein the network is at least one of an internet, an intranet, a wireless network, and a wired network.

11. The method according to claim 1, wherein at least one of the first information and the second information is received from a portable electronic device.

12. The method according to claim 11, wherein the portable electronic device is at least one of a handheld personal computer, a telephone, a mobile phone, a television set, and a personal organizer.

13. The method according to claim 1, further comprising:
selecting at least one subsequent question according to the diagnostic algorithm;
providing the at least one subsequent question; and
receiving subsequent information reflecting at least one answer to the at least one subsequent question.

14. The method according to claim 13, wherein the selecting and providing of at least one subsequent question continues until the determining of the diagnosis.

15. The method according to claim 1, wherein the diagnostic algorithm is a diagnostic algorithm generated by at least:
receiving, from a plurality of individuals, initial information reflecting answers to initial questions;
performing the multivariate analysis on the initial information received from the plurality of individuals to generate at least one synthetic variable; and
generating the diagnostic algorithm from at least the synthetic variable using the tree segmentation technique.

16. The method according to claim 1, wherein at least one of providing the first question and providing the second question comprises presenting, respectively, the first question or the second question at least one of over a network, at a kiosk, in a waiting room, at home, and at a point-of-sale.

17. The method according to claim 16, wherein the network includes at least one of an internet, an intranet, a wireless network, and a wired network.

18. The method according to claim 16, wherein the kiosk includes a display to present at least one of the first question and the second question.

19. The method according to claim 16, wherein the point-of-sale is at least one of a product-selling store, a service-providing location, and a website.

20. The method according to claim 1, wherein the diagnosis is at least one of a dermatological diagnosis, a beauty diagnosis, and a cosmetic diagnosis.

21. The method according to claim 1, wherein the diagnosis relates to at least one of a skin characteristic and a keratin characteristic.

22. The method according to claim 21, wherein the keratin characteristic relates to at least one of a hair, a nail, an eyelash, and an eyebrow.

23. The method according to claim 1, wherein the diagnosis relates to at least one of a skin condition and a keratin condition.

24. The method according to claim 23, wherein the keratin condition relates to at least one of a hair, a nail, an eyelash, and an eyebrow.

25. The method according to claim 23, wherein the skin condition includes at least one of greasy skin, dry skin, aging skin, wrinkled skin, marked skin, flask skin, squeamish skin, sensitive skin, skin phototype, a pigmented spot of skin, a problem with an eyelid, skin topography, a sensitive lip, a wrinkle around a lip, acne, and eczema.

26. The method according to claim 23, wherein the keratin condition includes at least one of hair loss, hair shine, hair thickness, hair oiliness, hair health, hair graying, and hair color.

27. The method according to claim 1, further comprising selecting at least one product according to, at least in part, the diagnosis.

28. The method according to claim 27, further comprising offering the product for sale.

29. The method according to claim 28, wherein the product includes at least one of a good and a service.

30. The method according to claim 28, wherein the product is a beauty product.

31. The method according to claim 1, further comprising providing at least one of advice and a recommendation according to, at least in part, the diagnosis.

32. The method according to claim 1, further comprising selecting at least one subject individual according to, at least in part, the diagnosis.

33. The method according to claim 32, wherein the subject individual exhibits a desired characteristic.

34. The method according to claim 33, wherein the desired characteristic is sensitive skin.

35. The method according to claim 34, further comprising evaluating a product on the subject individual.

36. A diagnostic method, comprising:
receiving a first question;
sending first information reflecting an answer to the first question;
receiving a second question, wherein the second question is a question selected according to the first information and according to a diagnostic algorithm generated using at least one of a multivariate analysis and a tree segmentation technique; and
sending second information reflecting an answer to the second question,
wherein a diagnosis is determined according to the diagnostic algorithm.

37. The method according to claim 36, wherein the multivariate analysis includes at least one of a principal component analysis, a factorial analysis, PLS path modeling, and structural equation modeling.

38. The method according to claim 36, wherein the tree segmentation technique includes a classification and regression tree method.

39. The method according to claim 38, wherein the classification and regression tree method is at least one of a CART method, a CHAID method, and a QUEST method.

40. The method according to claim 36, wherein the diagnostic algorithm is a diagnostic algorithm generated using at least both the multivariate analysis and the tree segmentation technique.

41. The method according to claim 40, wherein the multivariate analysis includes a principal component analysis and wherein the tree segmentation technique includes a classification and regression tree method.

42. The method according to claim 36, wherein the first question is a most discriminating question according to the tree segmentation technique.

43. The method according to claim 42, wherein the second question is, according to the tree segmentation technique and the answer to the first question, a second most discriminating question.

44. The method according to claim 36, wherein at least one of the first information and the second information is sent to a remote location over a network.

45. The method according to claim 44, wherein the network is at least one of an internet, an intranet, a wireless network, and a wired network.

46. The method according to claim 36, wherein at least one of the first information and the second information is sent by a portable electronic device.

47. The method according to claim 46, wherein the portable electronic device is at least one of a handheld personal computer, a telephone, a mobile phone, a television set, and a personal organizer.

48. The method according to claim 36, further comprising:
receiving at least one subsequent question selected according to the diagnostic algorithm; and
sending subsequent information reflecting at least one answer to the at least one subsequent question.

49. The method according to claim 48, wherein the receiving of at least one subsequent question continues until the diagnosis is determined.

50. The method according to claim 36, wherein the diagnostic algorithm is a diagnostic algorithm generated by at least:
receiving, from a plurality of individuals, initial information reflecting answers to initial questions;
performing the multivariate analysis on the initial information received from the plurality of individuals to generate at least one synthetic variable; and
generating the diagnostic algorithm from at least the synthetic variable using the tree segmentation technique.

51. The method according to claim 36, wherein at least one of the first question and the second question is received at least one of over a network, at a kiosk, in a waiting room, at home, and at a point-of-sale.

52. The method according to claim 51, wherein the network includes at least one of an internet, an intranet, a wireless network, and a wired network.

53. The method according to claim 51, wherein the kiosk includes a display to present at least one of the first question and the second question.

54. The method according to claim 51, wherein the point-of-sale is at least one of a product-selling store, a service-providing location, and a website.

55. The method according to claim 36, wherein the diagnosis is at least one of a dermatological diagnosis, a beauty diagnosis, and a cosmetic diagnosis.

56. The method according to claim 36, wherein the diagnosis relates to at least one of a skin characteristic and a keratin characteristic.

57. The method according to claim 56, wherein the keratin characteristic relates to at least one of a hair, a nail, an eyelash, and an eyebrow.

58. The method according to claim 36, wherein the diagnosis relates to at least one of a skin condition and a keratin condition.

59. The method according to claim 58, wherein the keratin condition relates to at least one of a hair, a nail, an eyelash, and an eyebrow.

60. The method according to claim 58, wherein the skin condition includes at least one of greasy skin, dry skin, aging skin, wrinkled skin, marked skin, flask skin, squeamish skin, sensitive skin, skin phototype, a pigmented spot of skin, a problem with an eyelid, skin topography, a sensitive lip, a wrinkle around a lip, acne, and eczema.

61. The method according to claim 58, wherein the keratin condition includes at least one of hair loss, hair shine, hair thickness, hair oiliness, hair health, hair graying, and hair color.

62. The method according to claim 36, further comprising selecting at least one product according to, at least in part, the diagnosis.

63. The method according to claim 62, further comprising buying the product.

64. The method according to claim 63, wherein the product includes at least one of a good and a service.

65. The method according to claim 63, wherein the product is a beauty product.

66. The method according to claim 36, further comprising receiving at least one of advice and a recommendation provided according to, at least in part, the diagnosis.

67. The method according to claim 36, further comprising receiving third information reflecting the diagnosis.

68. A method of generating a diagnostic algorithm, comprising:
receiving information reflecting a plurality of individuals' answers to questions;
performing an analysis on the received information to generate a synthetic variable; and
generating a diagnostic algorithm from at least the synthetic variable using a tree segmentation technique.

69. The method according to claim 68, wherein the method further comprises presenting, to a plurality of individuals, a questionnaire including the questions, and wherein receiving the information comprises collecting answers to questions of the questionnaire.

70. The method according to claim 69, wherein the questionnaire and the answers to the questions of the questionnaire are respectively presented and collected over a network.

71. The method according to claim 70, wherein the network is at least one of an internet, intranet, wireless network, and wired network.

72. The method according to claim 68, wherein the analysis is a multivariate analysis.

73. The method according to claim 72, wherein the multivariate analysis includes at least one of a principal component analysis, a factorial analysis, PLS path modeling, and structural equation modeling.

74. The method according to claim 73, wherein the principal component analysis is hierarchical.

75. The method according to claim 68, wherein the tree segmentation technique includes a classification and regression tree method.

76. The method according to claim 75, wherein the classification and regression tree method is at least one of a CART method, a CHAID method, and a QUEST method.

77. The method according to claim 68, wherein performing the analysis on the received information to generate a synthetic variable comprises:
selecting, from the received information, at least two groups of information reflecting answers that relate to differing sub-topics, respectively;
performing an analysis on each selected group of information to generate respective sub-topic variables; and
performing an analysis on the sub-topic variables to generate the synthetic variable.

78. The method according to claim 77, wherein the sub-topic variables correspond to a first factorial axis of a principal component analysis.

79. The method according to claim 77, wherein the sub-topics include at least one of atopy, sensitivity, cosmetic reactivity, environmental reactivity, and vascular reactivity.

80. The method according to claim 68, wherein the synthetic variable corresponds to a first factorial axis of a principal component analysis.

81. The method according to claim 68, wherein the diagnostic algorithm diagnoses at least an overall sensitivity of a subject individual.

82. The method according to claim 68, further comprising transforming the synthetic variable with a linear transformation.

83. The method according to claim 68, wherein generating the diagnostic algorithm using the tree segmentation technique includes:
selecting most discriminating questions; and
generating a limited set of the most discriminating questions that can provide a diagnosis by progressing through a tree.

84. The method according to claim 83, wherein the diagnostic is associated with a terminating branch in the tree.

85. The method according to claim 68, wherein the diagnostic algorithm provides at least one of a dermatological diagnosis, a beauty diagnosis, and a cosmetic diagnosis.

86. A system, comprising:
a data processor; and
a storage medium functionally coupled to the data processor, wherein the storage medium contains instructions to be executed by the data processor for performing the method according to claim 1.

87. A system, comprising:
a data processor; and
a storage medium functionally coupled to the data processor, wherein the storage medium contains instructions to be executed by the data processor for performing the method according to claim 36.

88. A system, comprising:
a data processor; and
a storage medium functionally coupled to the data processor, wherein the storage medium contains instructions to be executed by the data processor for performing the method according to claim 68.

89. A computer program product, comprising a computer-readable medium, wherein the computer-readable medium contains instructions for executing the method according to claim 1.

90. A computer program product, comprising a computer-readable medium, wherein the computer-readable medium contains instructions for executing the method according to claim 36.

91. A computer program product, comprising a computer-readable medium, wherein the computer-readable medium contains instructions for executing the method according to claim 68.
